(19) United States
(12) Patent Application Publication
(10) Pub. No.: US 2026/0232951 A1
(43) Pub. Date: Aug. 13, 2026

(54) Lexical Decision Task Command System for Sleep Apnea Remediation

(71) Applicants: Walt Froloff, Aptos, CA (US); Marcelo Siero, Scotts Valley, CA (US)

(72) Inventors: Walt Froloff, Aptos, CA (US); Marcelo Siero, Scotts Valley, CA (US)

(21) Appl. No.: 19/053,121

(22) Filed: Feb. 13, 2025

(51) Int. Cl. A61M 21/02; A61B 5/00

(52) U.S. Cl. CPC A61M 21/02; A61B 5/0026; A61B 5/4818

(57) ABSTRACT

A system for administering programmable lexical decision task commands to a sleeper upon detection of apnea episodes of breathing cessation is disclosed. The LDT commands sent are to restart breathing without waking sleep apnea suffer. The stimulus LDT commands are timed for behavioral responses. Snoring detect via an AI analytic and lexical decision task for cessation of snoring is also included.

### BACKGROUND

### Field of the Invention

The present invention generally relates to the field of sleep apnea remedies and more specifically to the use of lexical decision task aural commands issued to an apnea sufferer upon detection of apnea symptomatic breathing cessation and snoring.

### Technical Field

The present invention relates to an apparatus to detect and remedy prolonged breathing cessation symptomatic of sleep apnea during sleep, from respiration sounds, and specifically to initiate programmable customizable non-waking reminders to breathing upon set periods of detected extended breathing cessation.

### BACKGROUND

Sleep Apnea and Hypopnea are breathing disorders that occur during periods of sleep. Hypopnea is: a partial reduction in airflow during sleep. It's typically defined as a 30% to 50% reduction in airflow for at least 10 seconds, often accompanied by a decrease in blood oxygen saturation. While sleep apnea involves a complete or near-complete cessation of breathing, hypopnea is a partial reduction. Symptoms: Like sleep apnea and hypopnea can lead to disrupted sleep, daytime fatigue, and other health issues. It's usually diagnosed through a sleep study (polysomnography). Sleep apnea can be related to physical obstructions in the airway, neurological issues, or a combination of factors. Current treatment include lifestyle changes, CPAP (Continuous Positive Airway Pressure) therapy, or other interventions involving surgeries. Hypopnea and sleep apnea can significantly impact a person's health, quality of life and longevity if left untreated.

Most prior art for detecting the onset of a sleep cessation or normal exhale period, that rely on detecting the sounds of breathing, which can be confused by extraneous noises, coughing, wheezing and other internally generated biologic sounds. Although many mathematical and technical sophistication for identification and remediation without wakening the sleeper necessary to correctly identify more than normal breathing cessation has many solutions but heretofore still not been seen in the practical world of sleep apnea or prior art. The prior art is replete with data collection apps and many physical devices and can be found in the market place. However, these have no shown themselves as solutions to solve the sleep apnea problem for millions still burdened with this affliction.

In a rough estimate, at least 20-30 million people in the US have sleep apnea without an effective solution. It is estimated the up to 80% of moderate and severe obstructive sleep apnea, OSA, cases are undiagnosed. Even among the diagnosed not all receive effective treatment. The reasons are varied. Current sleep disorder healthcare is expensive and solutions are not conducive to physical implementation. What are needed are better and cheaper solutions, where treatments that are physically conducive to application by sufferers. This begins with detection.

In addition to breath signal sourcing, in order for both a microphones and remedial devices to work most effectively, they must be in proximity with a sleeper's mouth. This restriction or constraint has proven to be unacceptably uncomfortable to the sleeper. 50% of sleepers fail to keep a CPAP face device strapped to their face, unconsciously or unconsciously discarding the CPAP device during sleep and finding him or herself with the apparatus on the floor without a memory of disabling the nuisance. The comfort allowed by any sleeper's head or body mounted sleep apnea detection and remediation is critical. The field of sleep apnea relief is replete with prior art that does not meet these stringent conditions of sleeper's comfort or ease of use or customization to a sufferer.

Prior art for sleep apnea detection is replete with apparatus that must be strapped on or require sensors dependent on the sleeper's position while violating a sleeper's comfort and ease of use almost from the start. Adding apparatus installing instruction to guide the user on how to properly position the apparatus are not relevant as they will likely be discarded during sleep.

In other prior art, a 3-axis accelerometer determines the position of the user (supine, lateral, or dorsal). This positional information is evaluated by the processor embedded in the wearable apparatus or smart phone application in conjunction with the history of apneas, as determined by the processor embedded in the wearable apparatus or smart phone application from the audio signature of respirations collected by the microphone. Accelerometers and other sensors require physical connector and are generally an irritation and an anathema to sleepers that move around during sleep, causing an entanglements that produces discomfort and eventual discarding of the device by users. For example 50% of CPAP users discard the device at some point during sleep from discomfort and or irritations.

Even just an intermittent cessation or reduction of ventilation during sleep that results in a decrease in blood oxygen levels (hypoxia), increase in CO2 (hypercapnia), and vasoconstriction is unacceptable. An apnea is defined as a greater than 90% reduction in airflow for longer than 10 seconds. Hypopnea is overly shallow breathing a 30% or more reduction in airflow lasting 10 seconds or longer and can result in waking the sleeper, causing the lack of sleep necessary for normal sleep.

The causes of the various forms of sleep apnea and hypopnea are not fully understood and the current remedies are insufficient, leaving at least 33 million Americans with little or no solution to sleep loss and consequent adverse symptoms caused by sleep apnea and sleep disorders. The average apnea event lasts 20 seconds however events of 2 to 3 minutes are also known to occur. In addition any remedy must address the subjective nature of the illness as degrees of severity differ for each person. During the event, a number of physiological events also occur which are detrimental to good health.

Therefore, there is a need for an apparatus, system and method for detecting symptomatic sleep apnea, cessation of normal breathing, and for the issuing of non-waking stimulants to resume normal breathing. In addition, there is a need for an apparatus, system and method for detecting other apnea symptomatic characteristics with corresponding issuance of non-waking stimulants that are easy to use, comfortable, non-invasive and function without hindrance to the sleeper from a practical standpoint.

Some prior art require a very wide range of stimuli to resume breathing from a detected sleep apnea event. These analyze a multiplicity of parameters derived from redundant apparatus to detect respiration. Stimulation to resume respiration includes a multiplicity of embedded audio files and haptic pattern files, each with a distinct irritation, or amplitude of arousal index. This invention teaches that files produce the stimulus required to initiate an inhalation at the lowest amplitude stimulation. Since this prior art invention is based on the premise that there are many file combinations that will produce the stimulus required to initiate an inhalation at the lowest level of stimulation. Thus a multiplicity of embedded audio files and other stimulation sources are required. This approach conflicts with the requirements that a solution to sleep apnea should be is easy to use, comfortable, non-invasive and doesn't irritate the sleeper enough to cause the sleeper to discard the apparatus in their sleep, rendering this solution useless to at least some.

The key differences between regular breathing and snoring occur because: Regular breathing is controlled by smooth muscle movements, Snoring involves vibration of soft tissues, Air turbulence during snoring creates additional sound frequencies, Partial airway obstruction during snoring affects airflow patterns, Snoring typically shows more chaotic and variable patterns compared to the rhythmic pattern of normal breathing

Snoring sounds and regimes are typically rough and irregular compared to the quiet, smooth sound of normal breathing. What is needed are detection of snoring modes for the purposes of treatment that can differentiate from typical sleep apnea regimes for detection of snoring sounds which are more complex and irregular and not conducive to more conventional methods of signal processing.

Hence what is needed ways to identify the different causes of sleep apnea from a simple basic system and method using aural commands issued to sleepers detected to be exhibiting sleep apnea symptoms of breathing cessation beyond the cessation found in a normal sleep cycle. Currently, the conventional and traditional methods and devices leave 33 million Americans to fend for themselves on this debilitating and life shortening affliction.

Sleep has long been considered as a state of behavioral disconnection from the environment, without reactivity to external stimuli. Because behavioral responses have long been assumed to be possible only during wakefulness, they are generally rejected from the analysis or not collected at all in sleep studies. The rare studies that did measure behavioral responses in sleeping participants discovered manual behavioral responses during sleep onset, but not in deeper or other sleep stages.

A recent study¹ challenges the ‘sleep disconnection’ dogma requiring a multiplicity and complexity of parameter inputs for detection and remediation to prove out motor capacities. The new findings suggests that some level of environmental awareness and responsiveness persists during sleep. Participants in a study were given lexical decision task stimuli, instructed to frown or smile based on a simple and single verbal command. A lexical decision task is a procedure used in many psychology and psycholinguistics experiments. The basic procedure involves measuring how quickly sleepers classify stimuli as words or non-words. Although versions of the task had been used by researchers for a number of years, in the study these commands were received and carried out while participants maintained sleep. In short, behavioral and brain ¹“Behavioral and brain responses to verbal stimuli reveal transient periods of cognitive integration of the external world during sleep” Nature Neuroscience 12 Oct. 2023, Barak Turker, et al.
responses to verbal stimuli reveal transient periods of cognitive integration of the external world during sleep.

For real physiological reasons like muscle and brain function disconnection, sleep has classically been considered a state in which we cannot react to external stimuli. Unlike all previous sleep apnea studies used in prior art solutions, this most recent study from 2023 found that sleepers can transiently process external stimuli at a high cognitive level and behaviorally respond to them across most sleep stages. And “Sleeping participants can respond to verbal command stimuli”. Study results demonstrate that sleepers can perceive verbal stimuli, make a lexical decision and perform an adequate motor response while remaining asleep. What is needed are ways to stimulate breathing when it has stopped as a result of an identified sleep apnea event. What is needed are methods to properly assure that commands given a sleep apnea sufferer will be perceived and acted upon.

Participants in the 2023 study cited above were given a Lexical Decision Task, for example instructed aural commands to frown or smile. Results showed accurate behavioral responses, for example muscle contractions, in most sleep stages for all groups, except during slow-wave sleep in healthy volunteers. The findings show that even during genuine sleep, there are brief periods when individuals can react to certain verbal commands given from a lexical decision task.

In the same study behavioral and brain responses to stimuli revealed transient periods of cognitive integration of the external world during sleep. Sleep has long been considered as a state of behavioral disconnection from the environment, without reactivity to external stimuli. Mere conversation is insufficient to overcome a state of behavioral disconnection in sleep mode. Moreover recent studies show that by directly investigating behavioral responsiveness in volunteers engaged in a lexical decision task, results suggested that transient windows of reactivity to external stimuli exist during bona fide sleep. Such windows of reactivity pave the way for real-time communication with sleepers to probe sleep-related mental and cognitive processes. What is needed are ways to detect for precise instances and task those suffering from sleep apnea with non-waking stimuli to restart breathing where long pauses in breathing would otherwise prevail.

Because behavioral responses have long been assumed to be possible only during wakefulness, they are either rejected from the analysis or not collected at all in sleep studies. The recent study the 2023 cited above, found sleeping participants can respond to auditory stimuli. In that study, the participants' ability to behaviorally respond to auditory verbal stimuli across different sleep stages. Results showed that sleeping participants can respond to auditory stimuli. In this study they tested participants' ability to behaviorally respond to auditory verbal stimuli across different sleep stages and found that words and pseudowords audibly administered showed participants actually performed a lexical decision task while asleep. What is needed are systems which can deliver lexical decision tasks to sleepers with sleep apnea which instructs the sleepers to breath normally.

In an attempt to cover a large spread of sleep regimes and levels, some prior art source from multiple sensors for breathing signal. Breathing signal arrives from several “Initial Referential Parameters” and further calculation. These initial Referential Parameters are calculated from processed measured sets of the parameters and stored in memory for later use in arriving at breathing signal character. What is needed is simple single source real-time measured signals of sleeping user breath for stimuli so that response time is measured and responded with the sleeper's need for oxygen in time to cut short sleep apnea breathing cessation periods.

Sleep apnea is been documented as life shortening from 10-15 years. Sleep apnea can contribute to death from “natural causes” in several ways. The repeated oxygen deprivation and stress on the body from sleep apnea creates a cascade of harmful effects that can lead to death, both directly and indirectly. Direct mechanisms include: sudden cardiac arrest during sleep due to severe oxygen deprivation, fatal cardiac arrhythmias triggered by the stress of repeated breathing interruptions, and stroke due to decreased oxygen to the brain

A 10-15 year reduction in life expectancy comes primarily from these cumulative effects, particularly the cardiovascular complications. Each time you stop breathing during sleep: oxygen levels drop, the body releases stress hormones, blood pressure spikes, and heart rhythm becomes irregular. When this occurs repeatedly, night after night, it puts enormous strain on the cardiovascular system. Over time, this leads to chronic conditions that significantly increase mortality risk, hence death from “natural causes”.

What is needed is simple non-invasive non-intrusive systems that can restart sleep apnea extended delays in inhalation in real time when the sleep apnea breathing cessation episodes emerge, and without waking a sleeper. What is needed are simple aural Lexical Decision Task commands that are issued when a reminder to re-start breathing from an interruption or extended delay in inhalation is detected.

### SUMMARY

The present invention discloses a lexical decision task stimulation system for sleep apnea detection and treatment. This comprises a computer system having a processor, storage media, with at least one speaker and at least one microphone, wireless protocol and logic for processing breathing signal with at least one microphone configured to communicate acoustical source breathing signal to the computer system and the computer system is configured to communicate audible command to at least one speaker proximate to the acoustic breathing signal source.

The processor executes logic converting acoustic analog breath signal to raw digitized breath data stream transforming the digitized breath data stream into an enveloped breath data stream, averaging enveloped breath data stream inhalation-exhalation peaks for deriving a normalized enveloped breath data stream peak average amplitude and calculates a breath cessation demarcation amplitude. Verbal commands, in the Lexical Decision Task procedure are sent to a sleeper exceeding a preset max no breathing time period to resume inhalation at a preset acoustic volume to the speaker, and the cessation timer is incremented if non-responsive to a continued reading enveloped breath data stream with verbal command volume increase as per the Lexical Decision Task process. In short, a detection of breathing cessation signal beyond a preset time interval triggers a verbal command stimulation to resume breathing and a continued non-response for a preset delay time will re-trigger the verbal command to resume breathing until normal breathing signal amplitude is detected. Snoring is treated similarly, where detection of snoring will cause an verbal command to be issued to the snorer in the LDT process.

### BRIEF DESCRIPTION OF DRAWINGS

Specific embodiments of the invention will be described in detail with reference to the following figures.

FIG. 1 is a schematic of a sleep apnea treatment LDT system electronic hardware configuration in an embodiment of the invention.

FIG. 2 shows the typical digitized respiration signal plot 201 representing a sleeper's breath signal as sound amplitude or energy as a function of time.

FIG. 3 shows a transformed real-time breath data stream to enveloped breathing data stream in an embodiment of the invention.

FIG. 4 shows the logic for detection of breathing cessation for a measured preset time interval from onset of breathing out and issuance of LDT breathing resume command in an embodiment of the invention.

FIG. 5 Displays a logic flow diagram for Snoring detection and Lexical Decision Task stimulation remediation in an embodiment of the invention.

FIG. 6 illustrates a functional display UX for a programmable sleep lexical decision task device for functions provided in an embodiment of the invention.

### DETAILED DESCRIPTION

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

### Objects and Advantages

The present invention discloses an apparatus and method for control and detection of breathing cessation without sleep discomfort or waking. Devices for application of Lexical Decision Tasking are implemented for sleep apnea treatment.

An object of the invention is to use auditory lexical commands to restart breathing upon detection of delayed inhalation or exhalation.

Another object of the invention is to provide better and simpler breathing inhalation or exhalation cessation restart device that will not be irritating to the point to be discarded by the sleeper while sleeping.

Yet another object of the invention is to provide breathing restart reminder devices which are simple for users, have a friendly UX and provide user selected aural LDT to initiate normal breathing during sleep apnea air flow cessation events.

Another object of the invention is to provide breathing restart reminder devices which are of comfortable install and wear during sleep.

Another object of the invention is to provide a solution using small inexpensive computers capable of using the higher mathematic functions in signal processing to discover analog breathing signal characteristics and properties.

Yet another object of the invention is to apply auditory stimuli to sleep apnea sleepers at only operative periods and sequentially where the sleepers are initially non-responsive.

Another object of the invention is to provide powerful and insightful signal processing on analog acoustic signals to detect longer than normal sleep cycle breathing cessation.

Another object of the invention is to use lexical decision task (LDT) process which involves measuring the speed at which sleepers classify stimuli as words or non-word responses.

Yet another object of the invention is to detect breathing cessation, and programmable issue an LDT command from stored commands to resume breathing and to measure the response in realtime to assure that breathing has resumed.

Another object of the invention is to provide users with their own pre-selected lexical decision task commands.

Yet another object of the invention is to provide users with the capability to select the lexical decision task command voice and voice volume.

An object of the invention using AI analytics for detecting and identifying snoring regime sounds for the purposes of remediation.

Yet another object of the invention is to provide users with capability to create and select their own Lexical Decision Task commands for sleep mode regime advantage and treatment.

Another object of the invention is to provide single source real-time measured signals of sleeping user breath for stimuli so that response time is measured and responded with the sleepers need for oxygen in time to cut short sleep apnea breathing cessation periods.

### Embodiments of the Invention

Specific embodiments of the invention will be described in detail with reference to the following figures. In embodiment of the invention we wish to alter behavior in sleepers while they sleep. To motivate a sleeper to behaviorally respond to auditory verbal stimuli across different sleep stages without waking, we issue an auditory command which can be recognized by a sleeper for a particular behavior and measure the time of the command elicited response. This is known as Lexical Decision Task, LDT, procedure, method or process.

In an embodiment of the invention sleepers are presented, auditory, with words, logatimes, pseudowords or commands. Their task is to indicate, whether the presented stimulus command is understood via timed response detection. For example when a sleeper that has temporarily stopped breathing, sleep apnea, is issued an auditory command to breath responds by initiating breathing within a given time. This process is based on the reaction time from a sleepers command reception and response behavior while still in sleep mode measured through the response behavior.

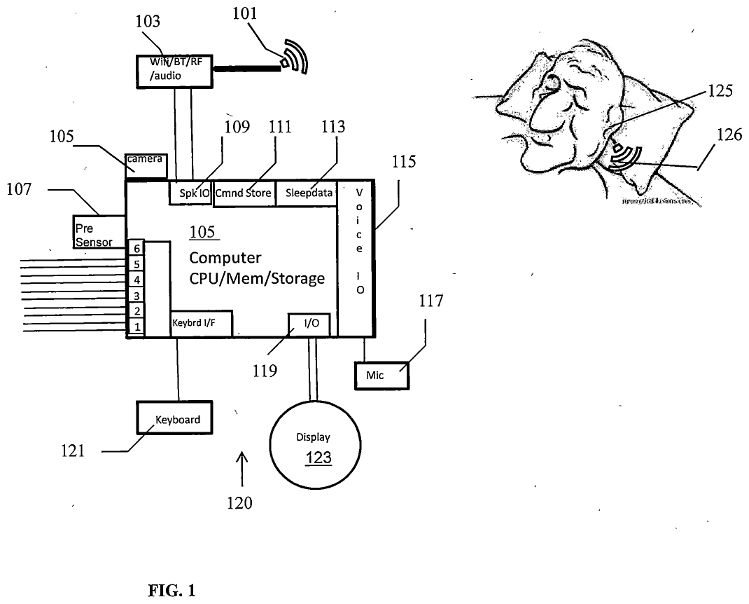

FIG. 1 is a schematic of a sleep apnea treatment LDT system electronic hardware configuration in an embodiment of the invention.

An embodiment of sleep apnea treatment system 120 includes a processor 105 electronically connected with supporting components, accessories, electronics and sensors. A multitasking O/S generally is used to coordinate and manage operations in accordance to system and programming software. A software development environment will typically provide a sophisticated mathematically resource libraries and available functions as the application to sleep apnea signal processing is required. In an embodiment, the schematic in FIG. 1 may represent a small computer system 105 as in a Raspberry Pi 105 or such with a keyboard 121 and display 123 unit and programmable processing power. Other such low cost embedded small computers as the ESP 32, Arduino, or cell phone may be used as well.

Acoustic breathing signal is sent by a sleeper 125 to either an earpiece 126 microphone or computer microphone 117 and with computer output to a speaker coupled communicatively with an audio signal receiving earpiece 126 speaker or in the computer speaker 103. The computer manages logic instructions from several peripherals comprising Wifi/BT/RF/voice circuits 103, antenna 101 LDT Command Storage 111, sleep data storage 113, voice i/o 115, display I/O 119, display 123, microphone 117, speakers 101, 125, keyboard 121, camera 105, telephone 109 and sensor I/O 107. These components become necessary to create the User Experience, UX, which provides users with the capability to manage their own experience and collected data from their sleep experience.

In an embodiment of the invention microphones 117, 126 and speakers 101, 125, are used to administer programmed auditory LDT commands and receive breathing signals to and from sleepers. In another embodiment, earbuds can be used for an integrated microphone and speaker. The earbud receives audio signals 125 and transmits 1126 breathing signal from sleeping user. The speaker communicates LDT commands or stimulus to a sleeping user from an LDT issuing computer system. The two devices are communicatively linked with LDT commands and breathing signals in response to LDT commands.

The computer system can be labeled or used interchangeably “Computer”, “microcontroller”, “processor”, and “smart phone” in any embodiment of the invention and are collectively defined as the device that relies on the application of software programs that are resident within the computer system. The limitations are that each is required to also have integrated operationally with at least one wireless protocol subsystem, and memory storage for storing and recalling LDT commands. The software environment operating the processor must contain libraries and executables for higher level math functions which are called routinely in processing of acoustical signal.

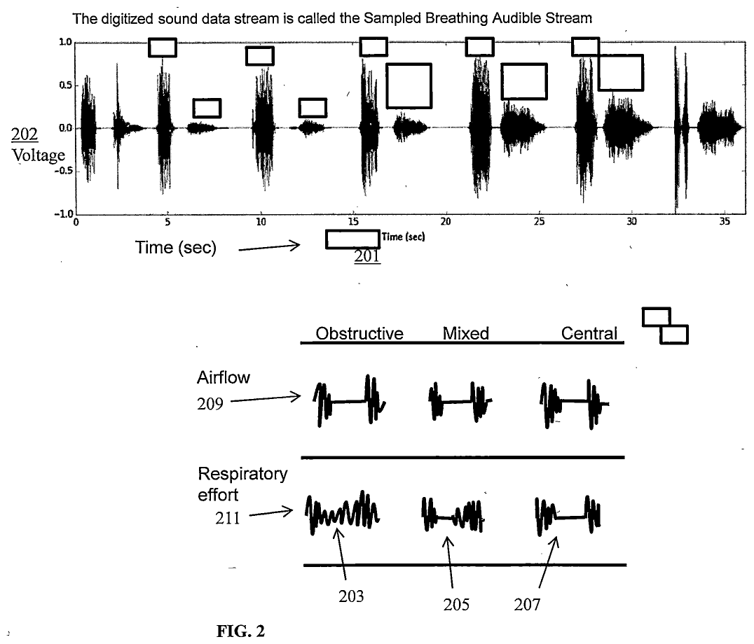

FIG. 2 shows the typical digitized respiration signal plot 201 representing a sleeper's breath signal as sound amplitude or energy as a function of time. There are 4-6 stages of sleep exhibiting varying and complex sound waveforms, modes and frequency content during sleep apnea. Sound signals in the human audible range, 20 Hz to 20 kHz, are the primary frequencies in consideration. Signal processing of the waveform and frequencies as shown, will need to accommodate certain sleep cycle intervals for the different stages and modes as Obstructive 203 Mixed 205 and Central 207 of sleep types to differentiate the actual breathing inhalation and non-breathing and exhalation periods of the signal waveforms processed.

In another embodiment sampling sleeper's acoustic breathing signals from an earbud device microphone, placed in a sleeper's ear, senses a sleeper's audible breathing signals and transmits the signal to the computer system. The sound signal amplitude generated by the patient's breathing whether from the sound of air flow 209 or respiratory effort 211 are processed through an analog to digital converter which converts analog to digital sampled data sequence which is transmitted to a computer processor as a sequence of data samples.

Digitizing an analog sound signal involves converting continuous sound waves into a discrete digital representation through a process called analog-to-digital conversion (ADC). This process consists of three main steps: sampling, quantization, and encoding. First, the analog signal is sampled at regular intervals, determined by the sampling rate (e.g., 44.1 kHz for CD-quality audio), capturing amplitude values at discrete points in time. Next, these sampled values are quantized, meaning they are mapped to the nearest value within a fixed range, typically determined by the bit depth (e.g., 16-bit, 24-bit), which affects the resolution and dynamic range of the digital audio. Finally, the quantized values are encoded as binary numbers, forming a digital audio file that can be processed, stored, and transmitted without degradation.

The sampling rate must be at least greater than the Nyquist criteria with margin. Typically the data sample sequence or data stream is transmitted to the computer wirelessly without encumbrance to the sleeper from an entanglement of wires, straps, facial gadgetry or body adhering sensors. In an embodiment of the invention wireless protocol technology such as Bluetooth or WiFi can be used to provide the communication between a sleeper and the processor device. The digitized sound data stream is called the Sampled Breathing Audible Stream 201. Disparate sleep apnea types, Central 207, Obstructive 203 and Mixed 205 sleep display different digitized data streams but have common characteristic inhalation, exhalation phases and cessation periods which for aspect of the invention will be classified as inhalation, exhalation and respiratory cessation representative of lack of airflow or respiratory effort.

### Processing the Sampled Breathing Acoustical Signal

In general, sampled audio signals have positive and negative voltage or amplitude values which are converted to a stream set of positive values. The waveform positive and negative voltage 202 is indicative and characteristic of scanning rate and not breathing rate. Therefore this raw sound data stream must be further processed to arrive and meaningful breathing type. The data stream depends on the signal scanning rate, the signal values are processed to represent an average signal amplitude envelope that carries the information corresponding to the detection of breath inhalations, exhalations and the time interval extending from exhalation, combined what we call cessation of breathing interval.

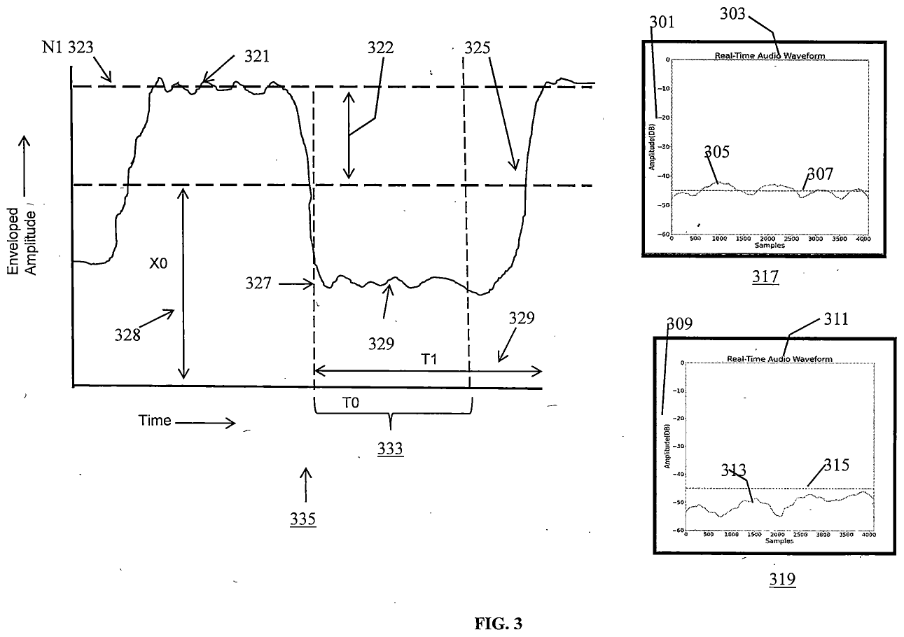

FIG. 3 shows a transformed real-time breath data stream to enveloped breathing data stream in an embodiment of the invention.

For acoustic signals in the human audible range, 20 Hz to 20 kHz, the primary considerations are for preserving the natural characteristics of the breathing data stream while effectively transforming the breath signal or processing data amplitude or sound energy envelope. The digitally converted analog signal shown in FIG. 2 201 is, depending on the received signal, sample rate creates a very jagged waveform which must retain its analog property energy characteristics through mathematical transformation to an acceptable enveloped waveform for digital identification of the inhalation, exhalation, and quiet phases in realtime. To accomplish this the scanned in breath stream data is buffered and uses mathematical transforms most suitable for smoothing and enveloping the scanned breath data stream 335.

As shown in an embodiment of the invention to detect a sleep apnea event, N1 is a individual peak average of a series of the enveloped breath data stream inhalation-exhalation amplitudes 321 and is used to determine a breath cessation demarcation amplitude X0 328 by subtracting a set default margin percentage 322 from the normalized average inhalation-exhalation peak amplitude N1. In another embodiment a breath cessation demarcation amplitude X0 can also be determined by a expert in sleep study data by inspection of the smoothing and enveloping the scanned breath data stream 335 and preset in the input variable parameter storage data.

An enveloped data stream transition 327 to below the X0 breath cessation demarcation amplitude X0 328, starts a clock for cessation of air flow 329. If a preset time period T0 333 is exceeded while amplitude 329 is still below the breath cessation demarcation amplitude 328 then a timer T1 330 is clocked starting at the preset default for allowed breathing cessation T0 to measure the time for a response to a command sent at T0. Commands to restart breathing can be continued until the onset of N1 inhalation-exhalation peak average amplitude 325 is attained.

The amplitude 301 309 or respiratory energy of the breath data stream wave form inhalation 317 and exhalation phase 319 are shown in enveloped data plot above 305 313 respectively. The cessation limit 307 and below 313 the cessation limit 315. Signal data stream waveform entry under the cessation limit triggers a clock and onset of a potential apnea episode. In an embodiment the enveloping mathematical operations on the raw breathing data stream derive function envelopes real-time audio waveform plots 303 311 used to calculate breathing in, breathing out and breathing cessation time intervals, from the instantaneous amplitude envelope of sound signal data stream. Data value cessation boundaries are derived from empirical data roughly measuring changes in breathing type energy or amplitude 301 309 and normalizing data values of enveloped breathing data stream to average peaks and drops in enveloped data stream values.

Transforming the instantaneous amplitude envelope of sound amplitude or energy from the raw data stream is done using mathematical filters such as the: Hilbert Transform, Rectification and Low-Pass Filtering, Moving Average, RMS, Wavelet Transform, Savitzky-Golay Filter, Exponential Moving Average (EMA), Teager-Kaiser Energy Operator, EMD (Empirical Mode Decomposition), and FFT-Based Envelope Detection. Any of these methods can be used to smooth out the scanned in data.

The different signal enveloping methods that can be used are described as: Moving Average—Ideal for smoothing amplitude variations; Rectification and Low-Pass Filtering—A straightforward and computationally efficient method to extract the amplitude envelope; Exponential Moving Average—Smoothes amplitude changes while being lightweight for real-time applications; Hilbert Transform: for calculating the instantaneous amplitude envelope of sound signal; FFT-Based Envelope Detection—for high-resolution envelope extraction in the frequency domain; Savitzky-Golay Filter—Preserves sharp transitions in the amplitude while smoothing.

In an embodiment, once scanned and digitized, breathing signal waveform formation is processing the breathing samples thru at least two stages of filtering. One stage can be an any of the above mathematical transform filters. A second stage filter is a low-pass filter, where not already done, which reduces the number of signal fluctuations, and reduces any signal temporal spikes which will otherwise corrupt or unevenly add error to the breathing signal information required, the audio sound energy in the amplitude.

As above described, in an embodiment of the invention an input sound signal is smoothed and filters the raw data stream, FIG. 2 201, an a resultant signal waveform is called a signal envelop. The positive and negative values and spikes of a raw input signal, are smoothed and averaged providing a measure of the average strength, energy or amplitude of the signal, regardless of its positive or negative fluctuations. This is done using one of the several mathematical enveloping transforms mentioned above. In an embodiment of the invention in includes taking the square root of the mean of a set of squared signal values creating an envelope of the smoothed representative signal. The smoothed out signal is then compared with a breathing cessation limit which is itself derived empirically from a period of enveloped signal averages having an average high amplitude energy at inhalation and also showing a low sound energy amplitude period representative of an initial exhalation period followed by an absence of breathing, ie breathing cessation.

In some embodiments the smoothed signal amplitude is converted to the values on a decibel 301 309 axis, which better represents the dynamic range of the enveloped breathing sound amplitude or energy 305 313 in a more scalable fashion and one more conducive for identifying the enveloped breathing signal changes from enveloped inhalation-exhalation 305 to cessation demarcation 313 amplitude. The smoothed signal of breathing energy or amplitude is then compared to an amplitude limit Ni set imperially by the identifying the breathing inhalation signal envelope average amplitude peaks and reducing that value by a margin representing the breathing cessation zone. With clocks the time interval to a resumption of breathing amplitude 305 triggered by the enveloped signal above the set amplitude value above 307 is used to establish a sleep apnea event or cessation of breathing for longer than a sleeper's normal non-breathing portion of the breathing cycle.

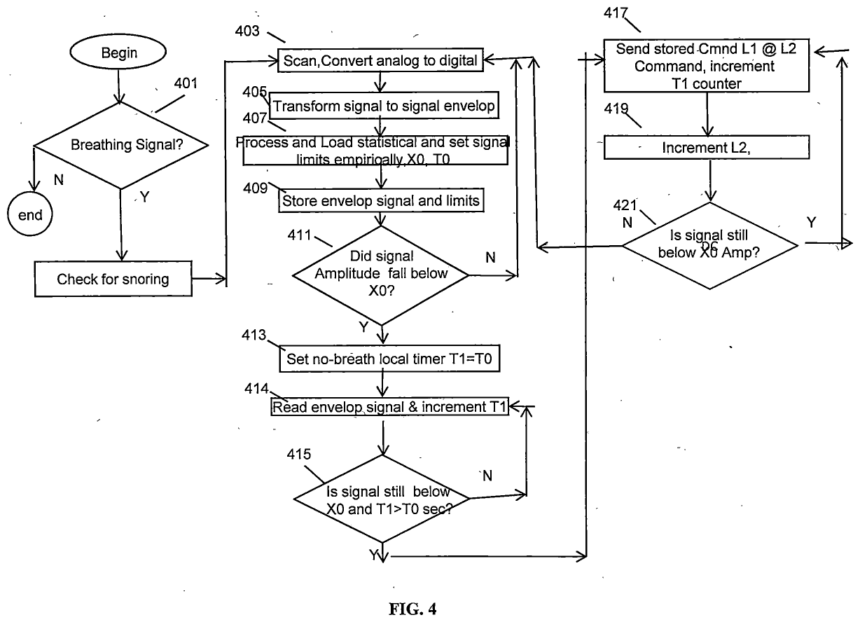

FIG. 4 shows the logic for detection of breathing cessation for a measured preset time interval from onset of breathing out and issuance of LDT breathing resume command in an embodiment of the invention.

Upon a reading of raw breathing signal 401 logic converts signal from analog to digital 403. A yes here means that the computer has power and a program is reading the earbuds, speakers, microphones, wireless and an acoustic signal is sensed. Logic transforms the sampled and digitized breath signal into a smooth signal envelop data stream 405 representing overall smoothed and averaged energy or amplitude of the signal. In an embodiment of the invention an option to check for snoring can be made. In an embodiment this enveloped breath signal can be buffered or stored and the enveloped breath signal is processed for empirically set 407 average inhale and exhale amplitude signal amplitude. At this point the signal amplitude is representative of the breathing signal energy having a two breathing phase cycle envelops of energy or amplitude values, one with an average higher amplitude, inhale-exhale phase and the other lower average amplitude, reduced breathing or cessation of breathing. On a given enveloped signal interval buffer the enveloped signal amplitude value is compared to the average amplitude value representative of the envelop and determined when the envelop signal drops below a set average enveloped signal amplitude level. This point in the signal indicates when breathing cessation occurs and its duration is measured to a time where the signal amplitude value cycles back to the proximate inhale-exhale breathing cycle phase demarcation amplitude average value. In this manner enveloped breathing signal amplitude values are compared and processed to smooth or average the breathing data stream amplitude values.

In an embodiment a low pass or band pass filter can act as an electronic circuit that allows signals with frequencies below a specific “cutoff frequency” to pass through while significantly reducing or eliminating signals with frequencies higher thereby reducing sharp spikes in the enveloped data stream which are not useful to contribute to the sound energy enveloped value sought to be markers for time of breathing cessation periods. Essentially filters reduce high-frequency components of a signal, and eliminates otherwise signal cluttering high frequency noise content from being interpreted as audible sound signal energy thereby increasing the fidelity to differentiate the breathing inhale-exhale signal amplitude from little or no-breathing signal breathing cessation periods.

In an embodiment of the invention earbud speakers are output device for issuing LDT commands to the sleeper when breathing has stopped for a longer than a set selectable “normal” period of time. As noted above, a lexical decision task, LDT, is a procedure which involves measuring how quickly people classify stimuli as words or non-words with acknowledged resulting behavior. In an embodiment of the invention upon detection of breathing cessation for a set period an LDT command issue starts a clock for measuring the response in realtime to assure that breathing has resumed.

The lexical decision task, LDT command has at least the following data parameters:
LDT command content: e.g. “Larry Breathe”, or “Breathe Now”, “Please Breathe”
Measured time interval from LDT issue to response: x seconds
Breathing cessation time: Preset_time_interval for no or low air flow period.

In some embodiments an LDT command volume or loudness is used to increase the command output to a sleeper where no response was registered.

LDT commands words and pseudowords are stored and programmably delivered or audibly administered to a sleeper experiencing sleep apnea of some kind or mode. Upon detection of breathing cessation beyond an allowed preset interval in the exhalation cycle, LDT command administering logic deliver lexical decision tasks to sleepers undergoing detected sleep apnea events. A local clock is started upon issuance of a LDT stimulation instructing a sleeper to resume breathing upon cessation beyond a preset minimum acceptable breathing inhalation-exhalation amplitude. The LDT command issued response time is also timed expiration of which prompts a re-issue of the LDT command.

In an embodiment the LDT command volume, command content, and breathing cessation time, are processed in compliance with adjusted statistically or empirically signal amplitudes for maximum signal fidelity, with LDT command issues during each iteration of continuous non-breathing no or low air flow cycle non-response periods.

The following is logic flow for a lexical decision task stimulation system for sleep apnea detection and treatment in an embodiment of the invention.

A computer system having a processor, storage media, with at least one speaker and at least one microphone, wireless protocol reads in breathing signal from a microphone configured to communicate acoustical breathing signal 401 from an acoustical breathing source to the computer system. A storage media configured for storing LDT commands and signal data. A processor executes logic responsive to the computer received 401 acoustical breath signal followed by sampling and conversion to digitized breath data stream 403. Processing logic for transforming digitized breath data stream into enveloped breath data stream 407, additionally logic is created for normalizing and averaging peak and trough enveloped breath data stream for measuring signal amplitude cessation-demarcation amplitude X0. In an embodiment some processing parameters as the X0, N1, T0 and or amplitude margin can be preset by inspection of sleep data. All data streams are saved and stored including all data stream, and cessation demarcation amplitude X0 409.

Logic provided is responsive to the onset of the enveloped data stream transition to below the cessation demarcation amplitude X0 411, if true start an cessation timer T1 413. Logic for continued reading enveloped breath data stream and incrementing cessation timer T1 continues to logic responsive to cessation timer T1 exceeding the preset time period T0 for normal air intake and enveloped data stream amplitude below the X0 cessation demarcation amplitude 415, where false the logic flows to continued reading 414 enveloped breath data stream and incrementing breathing cessation timer T1. The true branch logic flows to 417, to issue a stored lexical decision task command L1 to resume inhalation at a preset acoustic volume L2 is communicated to the speaker, and the cessation timer T1 is incremented.

Logic responsive to cessation timer T1 exceeding the T0 and enveloped data stream amplitude is below the X0 cessation demarcation amplitude 415, where false the logic loops back to continued reading breath data stream 403. Where the response is true the logic flows to 417, where a stored lexical decision task command L1 to resume inhalation at incremented acoustic volume L2 is sent to the speaker, and the cessation timer T1 is incremented.

Logic flow continues to incrementing acoustic volume L2 419 is and on to be responsive to signal amplitude above X0 and time T1 greater than T0 the cessation timer, T1 is incremented and checked for envelop signal amplitude below X0 low or no flow amplitude limit 421, branching to processing scanned and enveloped signal if breathing resumed. The true branch finds detection of above breathing cessation signal beyond a preset time interval triggering a verbal command stimulation to resume breathing 417 and a the false branch indicates continued response for a preset delay time to re-trigger the verbal command 417 to resume breathing until normal breathing signal amplitude is detected.

In short the detection of breathing cessation signal beyond a preset time interval triggers an LDT command stimulation to resume breathing and a continued non-response for a preset time interval will re-trigger the LDT command to resume until normal breathing signal amplitude response is detected.

In a simple scenario an average sleeper's cessation of breathing limit is preset for 20 seconds. At onset of breathing cessation, where the air flow falls below a signal energy determined amplitude, then a clock is started and if 20 seconds. If the preset cessation of breathing period of 20 seconds where the is exceeded while air flow falls below a signal energy determined amplitude, then an LDT command, eg “Larry breath”, is issued to a sleeper Larry and again, if there is no response in 2 seconds the LDT command to resume breathing is repeated and 10% louder. If still no response from an signal below a breathing cessation amplitude signal the LDT command to breath is repeated again and the loudness volume may be increased again.

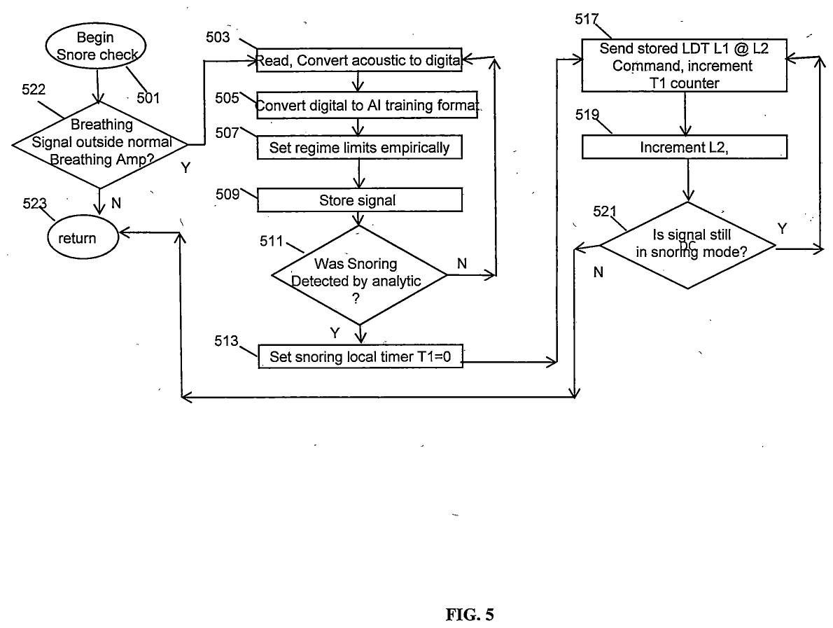

FIG. 5 Displays a logic flow diagram for Snoring detection and Lexical Decision Task stimulation remediation in an embodiment of the invention.

There are several sleep levels and breathing patterns. Snoring patterns are more irregular and can vary significantly, often show higher frequency oscillations (20-300 Hz). The frequency content contains multiple overlapping frequencies and show intermittent pauses. Their waveform characteristic are more complex, jagged waveform, with higher amplitude variations, sudden peaks and vibrations and often contains harmonics due to tissue vibration. This is why snoring sounds more rough and irregular compared to the quiet, smooth sound of normal breathing. Their detection is handled differently than the normal sleep pattern for apnea. For these and other reasons detection of snoring signal will take an alternate AI approach in an embodiment of the invention from signal processing.

### Snoring Detection

An AI analytics approach is an embodiment of the invention to create a sleep apnea system for detection of snoring. This embodiment, typical of AI analytic training models, will require audio data of both snoring and non-snoring sounds. The creation of training data collection from: audio snore samples from various individuals, son-snoring nighttime sounds and various environmental noises. The data is then: cleaned and segmented into the audio files, labeled as to the data containing snoring vs. non-snoring, and converted audio to a suitable format for machine learning (e.g., spectrograms or mel-frequency cepstral coefficients).

Two basic types of training data are used, positive and negative snoring samples. The positive samples include clear recordings of snoring from different people. The negative samples include: normal breathing sounds, environmental noises (e.g., fan noise, traffic sounds), other sleep-related sounds (e.g., coughing, turning in bed), variety in snoring types (e.g., soft, loud, intermittent, and continuous), and recordings from different microphone positions and room acoustics. These basic types of training data can be augmented from artificially created additional training samples by adding noise, changing pitch, or adjusting volume.

To create the application for implementation choose appropriate model architecture, e.g., CNN, RNN, or hybrid, and implement the model using a deep learning framework like TensorFlow or PyTorch. Proceed to process the data by: dividing the data into training, validation, and test sets, training the model on the prepared datasets and use techniques like cross-validation to ensure robust performance.

The model performance is evaluated on the test set and use metrics like accuracy, precision, recall, and F1-score. Then the model is integrated into an application implementing real-time audio processing for live snoring detection and in step labeled 411 for detection of snoring from the input signal.

There are available databases and datasets can be used for training a snoring detector. These datasets typically include audio recordings of snoring and other sleep-related sounds and sometimes include annotations for events such as snores, apnea events, or general sleep quality. Some options for training data sources include: PhysioNet Sleep Apnea Database, SLEEPEDFX Dataset, AudioSet, ESC-50 Dataset, CHIME-Snoring Dataset, Custom Data Collection.

Currently open source and proprietary software snoring detectors are available as well. Product 1: utilizes an ESP32 microcontroller to detect snoring sounds using artificial intelligence. It offers a code for implementation. Real-Time Snore Detector (RTSD): was developed using neural networks and selected sound features. SnoreLab: Several mobile phone app available for both iOS and Android platforms, records, measures, and tracks snoring.

In addition to the typical analytics approach to snoring detection above, AI transformers can be used for creating a snoring detection classifier, especially when combined with audio preprocessing techniques and well-annotated datasets. Snoring is a sequence-based phenomenon. Transformers can be used for capturing long-range dependencies in sequential data. Thus a transformer classifier analytic would identify the onset and offset times in detecting onset and cessation of a snoring regime sleep breathing data stream which is can be another embodiment to the invention.

Logic flow for LDT command stimulation for snoring detection.

A microphone configured to communicate acoustical breathing signal from an acoustical breathing source to the computer system and the computer system is configured to a speaker proximate to the acoustical breathing signal source 501. The processor executes logic responsive to the computer received 523 acoustical breath signal and conversion to scanned raw digitized breath data stream 503 which is converted to digital breath AI analytic training data format 505.

Breathing amplitudes are normalizing and averaged stream creating a statistical basis for amplitude cessation cycle 507 for a real-time storage of all data stream and cessation amplitude 509 data. The data stream is then processed by an AI snoring analytic 511, and if snoring is detected a snoring timer T1 513 is set and an lexical decision task is issued 517 at a preset volume to the acoustic signal source along with incrementing time counter T1. The volume L2 is incrementally increased and the logic is responsive to further signal detection for the snoring mode in the signal 521. If the snoring mode is still detected then a lexical decision task command is reissued at the incremented volume and the time T1 is incremented. This logic continues to loop until snoring signal ceases and loops back to reading the input signal 523. The detection of snoring triggers a LDT command stimulation to cease snoring and measures breathing signal amplitude and regime to insure compliance. Thus a lexical decision task stimulation system for sleep apnea detection and treatment includes detection of snoring and the lexical decision task command to remedy or cease snoring.

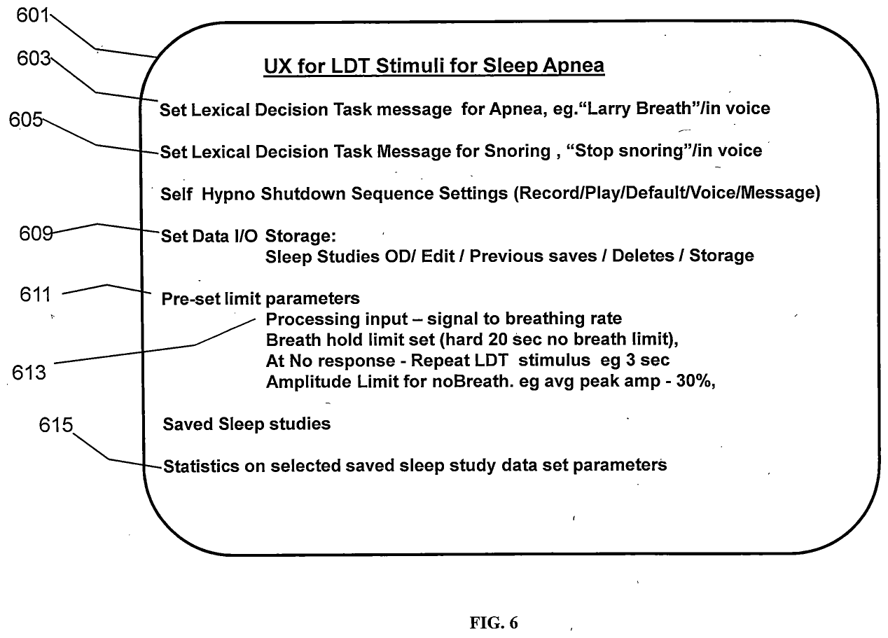

FIG. 6 illustrates a functional display UX for a programmable sleep lexical decision task device for functions provided in an embodiment of the invention.

UX can be for point and click, voice or touch screen displays. Since sleepers can transiently process external stimuli at a high cognitive amplitude and behaviorally respond while continuing to sleep across most sleep stages with LDT stimuli, LDT Lexical Decisions Task stimuli during sleep apnea episodes is designed to provide these functions and are employed in an device to treat sleep apnea disorders. These LDT stimuli can come in the form of verbal commands which need to selected, entered and stored for recall and use programmatically by a sleeper. A device UX is for awake users who wish to manage their own sleep apnea and snoring disorders, albeit with professional help and/or defaults.

Lexical Decision Tasks are often combined with technique called “priming”, and a participant is ‘primed’ with a certain stimulus before the actual lexical decision task has to be performed. This means that the UX must afford the capability to allow the user to custom create LDT commands. For example a user may wish to hear his/her own voice in the issuance of an LDT command. This can be recorded and used in the user's name, as in “Larry” followed by “breathe” or “Marcelo” before “stop snoring”. These “primers” for customized LDT commands will also be recorded and stored with and part of the stored LDT commands. The user may just as likely wish to use another, more authoritative voice in the issuance of the LDT command. LDT substitutes or defaults can be recorded and used as well. Thus an embodiment of the invention UX provides functionality for users to select, create and store their own LDT commands.

In some embodiments LDT command are controlled for their frequency and valence. Distinct lists of words and pseudowords are created and stored for programmable use during sleep apnea episodes. Thus functions on the UX are provided so that user's can store their preferences in voice and LDT command for application during their sleep periods and recalled programmatically as needed.

Some display 601 programmable functions include but not limited to: Set LDT message for Apnea in voice selection 603, Set LDT message for Snoring 605. Set Data I/O Storage 609 Sleep Studies OD/Edit/Previous saves/Deletes/Storage, Analysis on Stored data set, Processing input—signal to breathing rate 613, Breath hold limit set (eg, hard 20 sec nobreath detected), at No response-Repeat LDT stimulus eg 3 sec, Amplitude Limit for noBreath. eg avg peak amp—30% 613, Statistics on selected saved data set parameters 615

While the invention has been described with respect to a limited number of embodiments, those skilled in the art, having benefit of this invention, will appreciate that other embodiments can be devised which do not depart from the scope of the invention as disclosed herein. Other aspects of the invention will be apparent from the following description and the appended claims.

What is claimed is:

1. Lexical decision task stimulation system for sleep apnea detection and treatment comprising:
a computer system having a processor, storage media, with at least one speaker and at least one microphone, wireless protocol, and logic for processing breathing acoustic signals;
the at least one microphone configured to communicate an acoustical source breathing signal to the computer system and the computer system configured to communicate audible commands to at least one speaker proximate to the acoustic breathing signal source;
the processor executing logic responsive to the computer acoustic breath signal and conversion to digitized breath data stream;
logic for transforming the digitized breath data stream into an enveloped breath data stream;
logic for averaging enveloped breath data stream inhalation-exhalation peaks for deriving a normalized enveloped breath data stream inhalation-exhalation peak average amplitude N1 is used to calculate a breath cessation demarcation amplitude X0 by subtracting a set default margin percentage from the normalized average inhalation-exhalation peak amplitude N1;
logic for storage of all audible commands, signal data streams, normalized and breath cessation demarcation amplitude X0 data in the storage media;
logic responsive to the onset of the enveloped-data stream transition to below the breath cessation demarcation amplitude X0, and if true, starting a cessation timer T1 and if false, continued processing enveloped breath data stream and incrementing cessation timer T1;
logic responsive to cessation timer T1 exceeding a preset maximum no-breathing time period T0 and enveloped data stream amplitude below the X0 breath cessation demarcation amplitude, if true, then sending a stored verbal command L1 to resume inhalation at a preset acoustic volume L2 to the speaker, and the cessation timer T1 is incremented if false then continued reading enveloped breath data stream and incrementing cessation timer T1;
logic for incrementing the verbal command volume L2;
logic responsive to cessation timer T1 exceeding the T0 and enveloped data stream amplitude is still below the X0 cessation demarcation amplitude 415, the false branches the logic flow to 403 continued reading breath data stream, where true the logic flows to a branch for stored verbal command L1, which is sent to the sleeper to resume inhalation and incremented L2, and
logic incremented acoustic volume L2 419 is responsive to signal amplitude above X0 and time T1 greater then T0 and the cessation timer T1 is incremented and checked for envelop signal amplitude below X0 and T1
whereby detection of a breathing cessation signal beyond a preset time interval triggers a verbal command stimulation to resume breathing, breathing cessation signal beyond a preset time interval triggers a verbal command stimulation to resume breathing, and a continued non-response for a preset delay time will re-trigger the verbal command to resume breathing until normal breathing signal amplitude is detected.

2. The lexical decision task stimulation system for sleep apnea detection and treatment as in claim 1 further comprising a wireless earbud speaker with a microphone for communicating breathing signals remotely to the computer system having wireless protocol for coupled communication with the wireless earbud.

3. The lexical decision task stimulation system for sleep apnea detection and treatment as in claim 1 further comprising storing at least one lexical command.

4. The lexical decision task stimulation system for sleep apnea detection and treatment as in claim 1 further comprising the detection of snoring and sending lexical decision task command to remedy or cease snoring.

5. The lexical decision task stimulation system for sleep apnea detection and treatment as in claim 1 further comprising a user display/keyboard or voice interface whereby a user can create and store lexical commands.

6. The lexical decision task stimulation system for sleep apnea detection and treatment as in claim 1 further comprising retrieving an displaying sleep data from computer storage.

7. The lexical decision task stimulation system for sleep apnea detection and treatment as in claim 1 wherein the algorithm for enveloping signals is selected from a group of algorithms consisting of Hilbert Transform, Rectification and Low-Pass Filtering, Moving Average, RMS, Wavelet Transform, Savitzky-Golay Filter, Exponential Moving Average, Teager-Kaiser Energy Operator, Empirical Mode Decomposition, and FFT-Based Envelope Detection.

8. The lexical decision task stimulation system for sleep apnea detection and treatment as in claim 1 further comprising a trained AI analytic for the detection of snoring.

9. The lexical decision task stimulation system for sleep apnea detection and treatment as in claim 1 further comprising a user display UX with voice I/O.

10. A method for a lexical decision task stimulation system for sleeping disorder detection and treatment further comprising the steps of:
providing a computer system having a processor, storage media, with at least one speaker and at least one microphone, wireless protocol, and logic for processing breathing signals;
configuring at least one microphone to communicate the acoustical source breathing signal to the computer system, and the computer system is configured to communicate audible commands to at least one speaker proximate to the acoustic breathing signal source;
executing processor logic responsive to the computer source acoustic breath signal and conversion to digitized breath data stream;
logic for transforming the digitized breath data stream into an enveloped breath data stream;
logic for averaging enveloped breath data stream inhalation-exhalation peaks for deriving a normalized enveloped breath data stream inhalation-exhalation peak average amplitude N1 is used to calculate a breath cessation demarcation amplitude X0 407 by subtracting a set default margin percentage from the normalized average inhalation-exhalation peak amplitude N1;
logic for storing all verbal commands, signal data streams, normalized and breath cessation demarcation amplitude X0 data in the storage media;
logic responding to the onset of the enveloped-data stream transition to below the breath cessation demarcation amplitude X0, and if true, starting a cessation timer T1 and if false, continued processing enveloped breath data stream and incrementing cessation timer T1;
logic responding to cessation timer T1 exceeding a preset max no-breathing time period T0 and enveloped data stream amplitude below the X0 breath cessation demarcation amplitude, and if true, then sending a stored verbal command L1 to resume inhalation at a preset acoustic volume L2 to the speaker, and the cessation timer T1 is incremented, and if false, then continued reading enveloped breath data stream and incrementing cessation timer T1;
logic for incrementing the verbal command volume L2;
logic responding to cessation timer T1 exceeding the T0 and enveloped data stream amplitude is still below the X0 cessation demarcation amplitude 415, the false branches the logic flow to continued reading breath data stream, and where true the logic flows to a branch for stored verbal command L1, which is sent to the sleeper to resume inhalation and incremented L2, and
logic for incrementing acoustic volume L2 419 is responsive to signal amplitude above X0 and time T1 greater then T0 and the cessation timer T1 is incremented and checked for envelop signal amplitude below X0 and T1
whereby detection of a breathing cessation signal beyond a preset time interval triggers a verbal command stimulation to resume breathing and, a continued non-response for a preset delay time will re-trigger the verbal command to resume breathing until normal breathing signal amplitude is detected.

11. The method for a lexical decision task command system for sleep apnea detection and treatment as in claim 10 further comprising the step of providing a wireless earbud speaker with a microphone for communicating breathing signals remotely to the computer system having a wireless protocol for coupled communication with the wireless earbud.

12. The method for a lexical decision task command system for sleep apnea detection and treatment as in claim 10 further comprising the steps of providing a wireless earbud speaker with a microphone for communicating breathing signals remotely to the computer system having a wireless protocol for coupled communication with the wireless earbud.

13. The method for a lexical decision task command system for sleep apnea detection and treatment as in claim 10 further comprising the step of providing storage for at least one lexical command.

14. The method for a lexical decision task command system for sleep apnea detection and treatment as in claim 10 further comprising the step of providing logic for the detection of snoring and sending a lexical decision task command to remedy or cease snoring.

15. The method for a lexical decision task command system for sleep apnea detection and treatment as in claim 10 further comprising the step of providing a display/keyboard or voice interface for creating and storing lexical commands.

16. The method for a lexical decision task command system for sleep apnea detection and treatment as in claim 10 further comprising the step of providing logic and functionality for retrieving and displaying sleep data from computer storage.

17. The method for a lexical decision task command system for sleep apnea detection and treatment as in claim 10 wherein the algorithm for enveloping signals is selected from a group of algorithms consisting of Hilbert Transform, Rectification and Low-Pass Filtering, Moving Average, RMS, Wavelet Transform, Savitzky-Golay Filter, Exponential Moving Average, Teager-Kaiser Energy Operator, Empirical Mode Decomposition, and FFT-Based Envelope Detection.

18. The method for a lexical decision task command system for sleep apnea detection and treatment as in claim 10 further comprising the steps of providing logic for the detection of snoring via a trained AI analytic for the detection of snoring trained from snoring data.

19. The method for a lexical decision task command system for sleep apnea detection and treatment as in claim 10 further comprising the step of providing a device display UX with voice I/O.

20. Lexical decision task stimulation system for sleep snoring detection and treatment comprising:
a computer system having a processor, storage media, with at least one speaker and at least one microphone, wireless protocol, and logic for processing breathing signals;
the at least one microphone configured to communicate the acoustical source breathing signal to the computer system, and the computer system is configured to communicate audible commands to at least one speaker proximate to the acoustic breathing signal source;
the processor executes logic responsive to the computer-received acoustical breath signal and conversion to scanned raw digitized breath data stream converted to digital breath AI analytic training data format;
logic and processing for creating an AI analytic from AI analytic training models, at minimum using audio data of both snoring and non-snoring sounds for creating training data, which is formatted and segmented into the audio files, labeled as to the data containing snoring verses non-snoring, and converted audio to a suitable format for machine learning implementing the data through a model using a deep learning framework on the three datasets; training, validation, and tests;
executing logic for implementing the AI snore analytic via its API reading and responsive to the converted to digital breath stream, and if snoring is detected a snoring clock T1 is started, and a lexical decision task to cease snoring is issued at a preset audio volume to the acoustic signal source along with incrementing time counter T1;
logic for incrementally increasing audible volume responsive to further detection of snoring mode in the signal and re-issuing a lexical decision task command to cease snoring, measuring response time for signal updates in a loop fashion, and
logic for continuing signal detection of snoring triggering a command stimulation to cease snoring and measuring breathing signal amplitude for snoring cessation in the signal to ensure command compliance.
